(19) United States
(12) Patent Application Publication
(10) Pub. No.: US 2005/0222193 A1
(43) Pub. Date: Oct. 6, 2005

(54) Novel combination for the treatment of airway disorders

(76) Inventors: Guido Hanauer, KONSTANZ (DE); Wolfgang Kromer, Konstanz (DE); Stefan Postius, Konstanz (DE); Wolfgang-Alexander Simon, Konstanz (DE)

Correspondence Address: NATH & ASSOCIATES PLLC, 1030 FIFTEENTH STREET, N.W., SIXTH FLOOR, WASHINGTON, DC, 20005, US

(21) Appl. No.: 10/513,598

(22) PCT Filed: May 3, 2003
(86) PCT No.: PCT/EP03/04653

§ 371 Date: Nov. 5, 2004

(30) Foreign Application Priority Data
May 7, 2002 (EP) 02010305.7

(51) Int. Cl. A61K 31/4745; A61K 31/4439

(52) U.S. Cl. 514/292; 514/338

(57) ABSTRACT

The invention relates to the combination of reversible proton pump inhibitors and airway therapeutics for the treatment of airway disorders.

### TECHNICAL FIELD

The invention relates to the combination of certain known active compounds for therapeutic purposes.

### TECHNICAL BACKGROUND

A whole series of compounds are known from the prior art which inhibit gastric acid secretion by reversibly blocking the proton pump and which have therefore also been designated as reversible proton pump inhibitors (rPPI) or more recently as “APAs” (acid pump antagonists). These compounds are suitable for the treatment of gastric and intestinal disorders and reflux oesophagitis. Furthermore, compounds are known from the prior art which can be used for treating airway disorders and which are hereinbelow referred to as airway therapeutics. Their combined use in the meaning of the invention for therapeutic purposes has hitherto not been described in the prior art.

### PRIOR ART

International Patent Application WO 00/17200 describes certain tetrahydropyridoethers which are said to be suitable for the prevention and treatment of gastrointestinal diseases. In European Patent Application 259174 certain derivatives of 4-aminiquinolines are described which likewise are said to be useful in therapy for the inhibition of gastric acid secretion. In European Patent Application 774462 heterocyclic compounds of a certain formula are disclosed which are said to be useful for the prevention and/or treatment of bradykinin or its analogues mediated diseases in human being or animals. In International Patent Application WO 96/22978, substituted phenyl compounds are described which are said to be useful as endothelin antagonists. The combination of these compounds with compounds of a variety of other substance classes, inter alia with proton pump inhibitors, is mentioned. However, no particular utility of these combinations is given. In International Patent Application WO 98/16228 the combined use of a H⁺,K⁺-ATPase inhibitor and of a glucocorticoid in the treatment of asthma is described. International Patent Application WO 99/04816 relates to the combined use of a proton pump inhibitor and of an antibacterial active substance. International Patent Application WO 00/69438 describes inter alia the use of an NK-1 antagonist and a proton pump inhibitor in the preparation of a pharmaceutical composition for use in the treatment of asthma conditions. U.S. Pat. No. 6,060,472 relates to certain novel 3,5-disubstituted 1,2,4-thiadiazole compounds which are said to be effective in treating peptic ulcers by the inhibition of H⁺/K⁺-ATPase. T. O. Kiljander et al. (CHEST 1999; 116: 1257-1264) concluded after an 8-week double-blind, placebo-controlled crossover study with omeprazole as sole medication that there was a reduction in nocturnal asthma symptoms. W. J. Pan et al. (Aliment. Pharmacol. Ther. 2000; 14: 345-352) found a lack of pharmacokinetic interaction between lansoprazole or pantoprazole and theophyllin, without studying any effects of these combinations on asthma symptoms. J. Cuppoletti et al. (Clinical and Experimental Pharmacology and Physiology (2000) 27, 896-900) describe the activation of human ClC-2 Cl⁻ channels and the resulting implications for cystic fibrosis. D. Stancic-Rokotov et al. describe the beneficial effect of e.g. omeprazole on HCl-induced lung lesions in rats.

### DESCRIPTION OF THE INVENTION

Surprisingly, it has now been found that reversible proton pump inhibitors, whose original field of use is the treatment of gastric and intestinal disorders, are, in combination with airway therapeutics, particularly suitable for the treatment of airway disorders.

Accordingly, in a first aspect, the invention provides the combined use of reversible proton pump inhibitors and airway therapeutics for treating airway disorders.

Reversible proton pump inhibitors are designated as those substances which inhibit gastric acid secretion by blocking the proton pump, but which do not bind covalently to H+/K+-ATPase, the enzyme responsible for gastric acid secretion. According to the invention, the term “reversible proton pump inhibitor” includes not only the active compunds as such, but also their pharmacologically acceptable salts, solvates (in particular hydrates), etc.

Reversible proton pump inhibitors are described and claimed, for example, in the following patent applications and patents: EP 33094, EP 204285, EP 228006, EP 233760, EP 259174, EP 266326, EP 266890, EP 270091, EP 307078, EP 308917, EP 330485, U.S. Pat. No. 4,728,658, U.S. Pat. No. 5,362,743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029, WO 9928322, WO 9950237, WO 9951584, WO 9955705, WO 9955706, WO 0001696, WO 0010999, WO 0011000, WO 0017200, WO 0026217, WO 0029403, WO 063211, WO 0077003, WO 0158901, WO 0172754, WO 0172755, WO 0172756, WO 0172757, WO 0234749, WO 02060440, WO 02060441, WO 02060442, WO 03014120, WO 03014123 and WO 03016310, which are incorporated by reference into the specification in their entirety for all purposes.

Exemplary reversible proton pump inhibitors, which may be mentioned by their INNs or their codes are the compounds: AG-2000 (EP 233760), AU-461 (WO 9909029), BY112 (WO 9842707), Soraprazan (WO 0017200), CP-113411 (U.S. Pat. No. 5,362,743), DBM-819 (WO 0001696), KR-60436 (WO 9909029), Pumaprazole (WO 9418199), SKF-96067 (EP 259174), SKF-96356 (EP 307078), SKF-97574 (EP 330485), T-330 (EP 270091), T-776 (EP 270091), WY-27198 (U.S. Pat. No. 4,728,658), YH-1885 (WO 9605177), YJA-20379-8 (WO 9703074) and YM-19020 (EP 266890).

Among these, particular mention may be made of AU-461, Soraprazan, DBM-819, KR-60436, T-330, YH-1885 and YJA-20379-8.

Exemplary preferred reversible proton pump inhibitors, which may be mentioned are the compounds
(7R,8R,9R)-2,3-dimethyl-8-hydroxy-7-(2-methoxyethoxy)-9-phenyl-7,8,9,10-tetrahydroimidazo-[1,2-h]-[1,7]naphthyridine (Soraprazan),
(7R,8R,9R)-2,3-dimethyl-8-hydroxy-7-(2-methoxyethoxy)-9-phenyl-7H-8,9-dihydro-pyrano[2,3-c]-imidazo[1,2-a]pyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
7-(4-fluoro-benzyloxy)-2,3-dimethyl-1-(2-methyl-cyclopropylmethyl)-1H-pyrrolo[2,3-d]pyridazine,
7-(4-chloro-benzyloxy)-2,3-dimethyl-1-(2-methyl-cyclopropylmethyl)-1H-pyrrolo[2,3-d]pyridazine,
7-(2,4-difluoro-benzyloxy)-2,3-dimethyl-1-(2-methyl-cyclopropylmethyl)-1H-pyrrolo[2,3-d]pyridazine,
8-(2,6-dimethyl-benzylamino)-2,3-dimethyl-imidazo[1,2-a]pyridine-6-carboxylic acid (2-hydroxy-ethyl)-amide and
8-(2-ethyl-6-methyl-benzylamino)-2,3-dimethyl-imidazo[1,2-a]pyridine-6-carboxylic acid amide
   and the salts of these compounds.

The reversible proton pump inhibitors are present as such or in the form of their pharmacologically acceptable salts with bases or acids. Examples of salts with bases which may be mentioned are sodium, potassium, magnesium or calcium salts. Examples of salts with acids which may be mentioned are in particular water-soluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid oder 1-hydroxy-2-naphthoic acid, the bases or acids being employed in salt preparation—depending on whether it is a mono- or polybasic base or a mono- or polybasic acid and depending on which salt is desired—in an equimolar quantitative ratio or one differing therefrom.

If the reversible proton pump inhibitors or their salts are isolated in crystalline form, the crystals may contain variable amounts of solvent. Thus, according to the invention, the term “reversible proton pump inhibitor” also includes all solvates, in particular all hydrates, of the reversible proton pump inhibitors and their salts.

Airway therapeutics which are suitable for the purpose of the invention are active compounds from different classes of active compounds—with the exception of glucocorticoides in general, except ciclesonide, such as, for example, the following:
β₂-adrenoceptor agonists (in particular selectively acting substances having only slight cardiac action which, as a result, are also suitable for use in the therapy of airway disorders), such as, for example,
4-hydroxy-7-[2-[2-[3-(2-phenylethoxy)propoxy]ethylamino]ethyl]benzothiazol-2(3H)-one (AR-C68164AA),
3-[2-(4-hydroxy-2-oxo-2,3-dihydrobenzothiazol-7-yl)ethylamino]-N-[2-[2-(4-methylphenyl)ethoxy]ethyl]-propanesulphonamide (AR-C89855M),
5-[2-[N-(dimethylaminocarbonyl)-N-(1,1-dimethylethyl)amino]-1-hydroxyethyl]-1,3-benzenediol (BAMBUTEROL),
4-methylbenzoic acid 4-[2-[(1,1-dimethylethyl)amino]-1-hydroxyethyl]-1,2-phenylene ester (BITOLTEROL),
3-bromo-α-[(tert-butylamino)methyl]-5-isoxazolemethanol (BROXATEROL),
[5-[2-[(1,1-dimethylethyl)amino]-1-hydroxyethyl]-2-hydroxyphenyl]urea (CARBUTEROL),
4-[2-(6-phenethylaminohexylamino)ethyl]benzene-1,2-diol (DOPEXAMINE),
N-(3,3-diphenylpropyl)-α-methylcyclohexaneethylamine (DROPRENILAMINE),
(+/−)-2′-hydroxy-5′-[(RS)-1-hydroxy-2-[[(RS)-p-methoxy-α-methylphenethyl]amino]ethyl]formanilide (FORMOTEROL),
(R)-α₁-[(tert-butylamino)methyl]-4-hydroxy-m-xylene-α,α′-diol (LEVOSALBUTAMOL),
4-amino-3-chloro-α-[[(1,1-dimethylethyl)amino]methyl]-5-(trifluoromethyl)benzenemethanol (MABUTEROL),
(−)-(R)-2-(tert-butylamino)-1-(2-chloro-4-hydroxyphenyl)ethanol (MELUADRINE),
(+/−)-5,6-diisobutyryloxy-2-(methylamino)-1,2,3,4-tetrahydronaphthalene (NOLOMIROLE),
(RS)-{6-[2-(tert-butylamino)-1-hydroxyethyl]-3-hydroxy-2-pyridyl}methanol (PIRBUTEROL),
7-[3-[[2-(3,5-dihydroxyphenyl)-2-hydroxyethyl]amino]propyl-3,7-dihydro-1,3-dimethyl-1H-purine-2,6-dione (REPROTEROL),
α(1)-[[(1,1-dimethylethyl)amino]methyl]-4-hydroxy-1,3-benzenedimethanol (SALBUTAMOL),
(+/−)-N-[2-hydroxy-2-[4-hydroxy-3-(hydroxymethyl)phenyl]ethyl]-N-[6-(4-phenylbutoxy)hexyl]amine (SALMETEROL),
4-hydroxy-7-[2-[2-[3-(2-phenylethoxy)propysulphonyl]ethylamino]ethyl]benzothiazol-2(3H)-one (SIBENADET),
[R-(R*,R*)]-8-hydroxy-5-[1-hydroxy-2-[2-(4-methoxyphenyl)-1-methylethylamino]ethyl]-2(1H)-quinoline (TA-2005),
5-[2-[(1,1-dimethylethyl)amino]-1-hydroxyethyl]-1,3-benzenediol (TERBUTALINE),
5-chloro-3-[4-(2-hydroxyethyl)-1-piperazinyl]carbonylmethyl-2-benzothiazolinone (TIARAMIDE) and α-[(tert-butylamino)methyl]-o-chlorobenzyl alcohol (TULOBUTEROL);
muscarinic receptor antagonists, such as, for example,
endo-8-(2-fluoroethyl)-3-[(hydroxydiphenylacetyl)oxy]-8-methyl-8-azoniabicyclo[3.2.1]octane bromide (FLUTROPIUM BROMIDE),
3-(3-hydroxy-2-phenylpropanoyloxy)-8-isopropyl-8-methyl-8-azoniabicyclo[3.2.1]octane bromide (IPRATROPIUM BROMIDE),
(8r)-6β-7β-epoxy-8-ethyl-3-α-hydroxy-1-αH-5-αH-tropanium bromide (OXITROPIUM BROMIDE),
(R)-3-quinuclidinyl-(S)-β-hydroxy-α-[2-(R)-methylsulphiny]ethyl]hydratropate (REVATROPATE) and
[7(S)-(1α,2β,4β,5α,7β)]-7-[2-hydroxy-2,2-di(2-thienyl)acetoxy]-9,9-dimethyl-3-oxa-9-azoniatricyclo[3.3.1.0(2,4)]nonane bromide (TIOTROPIUM BROMIDE);
theophylline-like bronchodilators, such as, for example,
3,7-dihydro-1,3-dimethyl-1H-purine-2,6-dione/1,2-ethanediamine (AMINOPHYLLINE),
3,7-dihydro-1,3-dimethyl-7-[(5-methyl-1,2,4-oxadiazol-3-yl)methyl]-1H-purine-2,6-dione (CH INOIN-170),
7-(2,3-dihydroxypropyl)-1,2,3,6-tetrahydro-1,3-dimethylpurine-2,6-dione (DIPROPHYLLINE),
7-(1,3-dioxolan-2-ylmethyl)-3,7-dihydro-1,3-dimethyl-1H-purine-2,6-dione (DOXOFYLLI NE),
[R-(R*,S*)]-3-[(2-hydroxy-1-methyl-2-phenylethyl)amino]-1-(3-methoxyphenyl)-1-propanone (OXYFEDRINE),
3,7-dimethyl-1-hexyl-1H,3H-purine-2,6-dione (PENTIFYLLINE),
3,7-dihydro-3,7-dimethyl-1-(5-oxohexyl)-1H-purine-2,6-dione (PENTOXIFYLLINE),
3,7-dihydro-3-methyl-1-(5-oxohexyl)-7-propyl-1H-purine-2,6-dione (PROPENTOFYLLINE),
3,7-dihydro-7-(2-hydroxypropyl)-1,3-dimethyl-1H-purine-2,6-dione (PROXYPHYLLINE) and
3,7-dihydro-1,3-dimethyl-1H-purine-2,6-dione (THEOPHYLLINE);
PDE3/4- and PDE4 inhibitros, such as, for example, the compounds mentioned as examples in the following patent applications and patents:
EP 0163965, EP 0389282, EP 0393500, EP 0435811, EP 0482302, EP 0499216, EP 0506194, EP 0510562, EP 0528922, EP 0553174, EP 0731099, WO 9319749, WO 9500516, WO 9501338, WO 9600218, WO 9603399, WO 9611690, WO 9636625, WO 9636626, WO 9723457, WO 9728131, WO 9735854, WO 9740032, WO 9743288, WO 9809946, WO 9807715, WO 9808841, WO 9821207, WO 9821208, WO 9821209, WO 9822453, WO 9831674, WO 9840382, WO 9855481, WO 9905111, WO 9905112, WO 9905113, WO 9931071, WO 9931090, WO 9947505, WO 9957115, WO 9957118, WO 9964414, WO 0001695, WO 0012501, WO 0042017, WO 0042018, WO 0042019, WO 0042020, WO 0042034, WO 0119818, WO 0130766, WO 0130777 and WO 0151470, in particular the compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
3-(benzyloxy)-1-(4-fluorobenzyl)-N-[3-(methylsulphonyl)phenyl]-1H-indole-2-carboxamide,
(1S-exo)-5-[3-(bicyclo[2.2.1]hept-2-yloxy)-4-methoxyphenyl]tetrahydro-2(1H)-pyrimidinone (ATIZORAM),
N-(3,5-dichloro-4-pyridinyl)-2-[1-(4-fluorobenzyl)-5-hydroxy-1H-indol-3-yl]-2-oxoacetamide (AWD-12-281),
β-[3-(cyclopentyloxy)-4-methoxyphenyl]-1,3-dihydro-1,3-dioxo-2H-isoindole-2-propanamide (CDC-801),
N-[9-methyl-4-oxo-1-phenyl-3,4,6,7-tetrahydropyrrolo[3,2,1-jk][1,4]benzodiazepin-3(R)-yl]pyridine-4-carboxamide (CI-1018),
cis-[4-cyano-4-(3-cyclopentyloxy-4-methoxyphenyl)cyclohexane-1-carboxylic acid (CILOMILAST),
8-amino-1,3-bis(cyclopropylmethyl)xanthine (CIPAMFYLLINE),
N-(2,5-dichloro-3-pyridinyl)-8-methoxy-5-quinolinecarboxamide (D-4418),
5-(3,5-di-tert-butyl-4-hydroxybenzylidene)-2-iminothiazolidin-4-one (DARBUFELONE),
2-methyl-1-[2-(1-methylethyl)pyrazolo[1,5-a]pyridin-3-yl]-1-propanone (IBUDILAST),
2-(2,4-dichlorophenylcarbonyl)-3-ureidobenzofuran-6-yl methanesulphonate (LIRIMILAST),
(−)-(R)-5-(4-methoxy-3-propoxyphenyl)-5-methyloxazolidin-2-one (MESOPRAM),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (PUMAFENTRINE),
3-(cyclopropylmethoxy)-N-(3,5-dichloro-4-pyridyl)-4-(difluoromethoxy)benzamide (ROFLUMILAST) the N-oxide of ROFLUMILAST,
(RS)-4-(3-cyclopentyloxy-4-methoxyphenyl)-2-pyrrolidinone (ROLIPRAM),
5,6-diethoxybenzo[b]thiophene-2-carboxylic acid (TIBENELAST),
2,3,6,7-tetrahydro-2-(mesitylimino)-9,10-dimethoxy-3-methyl-4H-pyrimido[6,1-a]isoquinolin-4-one (TREQUINSIN) and
3-[[3-(cyclopentyloxy)-4-methoxyphenyl]methyl]-N-ethyl-8-(1-methylethyl)-3H-purine-6-amine (V-11294A);
prostaglandin D₂ antagonists, such as, for example,
(1R,2R,3S,5S)-7-[2-(5-hydroxybenzothiophen-3-ylcarboxamido)-6,6-dimethylbicyclo[3.1.1]hept-3-yl]-5(Z)-heptenoic acid (S-5751);
adenosine A₃ antagonists, such as, for example,
3-ethyl 5-(3-methylbenzyl)2-methyl-6-phenyl-4-(phenylethynyl)-1,4-dihydropyridine-3,5-dicarboxylate (MRS-1328),
propyl 6-ethyl-5-(ethylsulphanycarbonyl)-2-phenyl-4-propyl-pyridine-3-carboxylate (MRS-1523),
ethyl 6-ethyl-5-(ethylsulphanylcarbonyl)-2-phenyl-4-propylpyridine-3-carboxylate (MRS-1476),
propyl 2-(3-chlorophenyl)-4,6-diethyl-5-(propylsulphanylcarbonyl)-pyridine-3-carboxylate (MRS-1505),
ethyl 4-ethyl-5-(ethylsulphanylcarbonyl)-2-phenyl-6-propylpyridine-3-carboxylate (MRS-1486) und
cis-3-(5,6-dimethyl-2-phenyl-7H-pyrrolo[2,3-d]pyrimidin-4-ylamino)cyclopentanol (CDS-90910);
bradykinin B2 antagonists, such as, for example,
D-arginyl-L-arginyl-L-prolyl-L-(4-hydroxy)prolyl-glycyl-L-(2-thienyl)alanyl-L-seryl-D-(1,2,3,4-tetrahydroisoquinolin-3-ylcarbonyl)-(N-cyclohexyl)glycyl-L-arginine (CP-0597),
(E)-N-[N-[3-(3-bromo-2-methylimidazo[1,2-a]pyridin-8-yloxymethyl)-2,4-dichlorophenyl]-N-methylcarbamoylmethyl]-4-(N,N-dimethylcarbamoyl)cinnamamide (FR-167344),
3-(6-acetamido-3-pyridyl)-N-[N-[2,4-dichloro-3-(2-methylquinolin-8-yloxymethyl)phenyl]-N-methylcarbamoylmethyl]-2(E)-propenamide (FR-173657),
D-arginyl-arginyl-prolyl-[4(R)-hydroxy]prolyl-glycyl-(2-thienyl)alanyl-seryl-[1,2,3,4-tetrahydroisoquinolin-3(R)-ylcarbonyl]-[(3aS,7aS)-octahydroindol-2(S)-ylcarbonyl]-arginine (ICATIBANT),
1-[4-(aminoiminomethyl)benzoyl]4-[[(2S)-1-[[2,4-dichloro-3-[[(2,4-dimethyl-8-quinolinyl)oxy]methyl]-phenyl]sulphonyl]-2-pyrrolidinyl]carbonyl]piperazine (LF-16.0335) and
D-arginyl-L-arginyl-L-prolyl-L-(trans-4-hydroxy)prolyl-glycyl-L-phenylalanyl-L-seryl-D-(trans-4-propoxy)prolyl-L-[(2α,3β,7β)octahydroindol-2-ylcarbonyl]-L-arginine (NPC-17731);
leukotriene LTB₄ antagonists, such as, for example,
N-(ethoxycarbonyl)-4-[3-[4-[1-(4-hydroxyphenyl)-1-methylethyl]phenoxymethyl]benzyloxy]benzene-carboximidamide (AMELUBANT),
2-[3-[3-(5-ethyl-4′-fluoro-2-hydroxybiphenyl-4-yloxy)propoxy]-2-propylphenoxy]benzoic acid (LY-293111) and
4-[5-(4-amidinophenoxy)pentyloxy]-N,N-diisopropyl-3-methoxybenzamide (MOXILUBANT);
cysteinyl-leukotriene, receptor antagonists, such as, for example,
9-[(4-acetyl-3-hydroxy-2-n-propylphenoxy)methyl]-3-(1H-tetrazol-5-yl)-4H-pyrido[1,2-a]pyrimidin-4-one (AS-35),
(+)-4(S)-(4-carboxyphenylthio)-7-[4-(4-phenoxybutoxy)phenyl]-5(Z)-heptenoic acid (BAY-X-7195),
(E)-4-[3-[2-(4-cyclobutylthiazol-2-yl)viny]phenylamino]-2,2-diethyl-4-oxobutanoic acid (CINALUKAST),
6-(2-cyclohexylethyl)-[1,3,4]thiadiazolo[3,2-a]1,2,3-triazolo[4,5-d]pyrimidin-9(1H)-one (DS-4574),
7-[(1R,2S)-10-(4-acetyl-3-hydroxy-2-propylphenoxy)-1-hydroxy-1-(3-trifluoromethylphenyl)deca-3(E),5(Z)-dien-2-ylthio]-4-oxo-4H-1-benzopyrane-2-carboxylic acid (IRALUKAST),
4-[6-acetyl-3-[3-(4-acetyl-3-hydroxy-2-propylphenylthio)propoxy]-2-propylphenoxy]butyric acid (KCA-757),
4-[3-(4-acetyl-3-hydroxy-2-propylphenoxy)propylsulphonyl]-gamma-oxobenzenebutyric acid (L-648051)
(E)-2,2-diethyl-3′-[2-[2-(4-isopropyl)thiazolyl)ethenylsuccinanilinic acid (MCI-826),
2-[1-[1 (R)-[3-[2(E)-(7-chloroquinolin-2-yl)vinyl]phenyl]-3-[2-(1-hydroxy-1-methylethyl)phenyl]propylsulphanylmethyl]cyclopropyl]acetic acid (MONTELUKAST),
8-[4-(4-phenylbutoxy)benzamido]-2-(tetrazol-5yl)-4H-1-benzopyran-4-one (PRANLUKAST),
2(S)-hydroxy-3(R)-(2-carboxyethylthio)-3-[2-(8-phenyloctyl)-phenyl]propionic acid (POBILUKAST),
5-[2-[4-(quinolin-2-yl)methoxyphenoxymethyl]benzyl]tetrazole (RG-12525),
5-[3-[3-(2-quinolinylmethoxy)phenoxy]propyl]-1H-tetrazole (RG-7152),
1,1,1-trifluoro-N-[3-(2-quinolinylmethoxy)phenyl]methanesulphonamide (RITOLUKAST)
(1S,2R)-5-[3-[2-(2-carboxyethylthio)-1-hydroxypentadeca-3(E),5(Z)-dienyl]phenyl}-1H-tetrazole (SULUKAST),
2′-hydroxy-3′-propyl-4′-[4-(1H-tetrazol-5-yl)butoxy]acetophenone (TOMELUKAST),
5-[3-[2-(7-chloroquinolin-2yl)vinyl]phenyl}-8-(dimethylcarbamoyl)-4,6-dithiaoctanoic acid (VERLUKAST),
[[5-[[3-(4-acetyl-3-hydroxy-2-propylphenoxy)propyl]thio]-1,3,4-thiadiazol-2-yl]thio]acetic acid (YM-638),
4-(5-cyclopentyloxycarbonylamino-1-methylindol-3-yl-methyl)-3-methoxy-N-o-tolylsulphonylbenzamide (ZAFIRLUKAST) and
1(R)-3-methoxy-4-[1-methyl-5-[N-(2-methyl-4,4,4-trifluorobutyl)carbamoyl]indol-3-ylmethyl]-N-(2-methylphenylsulphonyl)benzamide (ZD-3523);
leukotriene synthesis inhibitors, such as, for example,
(+)-N-[3-[5-(4-fluorophenoxy)-2-furyl]-1(R)-methyl-2-propynyl]-N-hydroxyurea (ABT-175),
(R)-N-[3-[5-(4-fluorobenzyl)thien-2-yl]-1-methyl-2-propynyl]-N-hydroxyurea (ATRELEUTON),
(R)-2-cyclopentyl-2-[4-(quinolin-2-ylmethoxy)phenyl]acetic acid (BAY-X-1005),
(−)-2(R)-cycloheptyl-2-[4-(2-quinolylmethoxy)phenyl]-N-(methylsulphonyl)acetamide (BAY-Y-1015),
N-(3-phenoxycinnamyl)acetohydroxamic acid (BWA-4C),
(2S,5S)-trans-2-(4-fluorophenoxymethyl)-5-(4-N-hydroxyureidyl-1-butynyl)tetrahydrofuran (CMI-977),
(+/−)-4-(p-fluorobenzyl-2-(hexahydro-1-phenethyl-1H-azepin-4-yl)-1(2H)-phthalazinone (FLEZELASTINE),
1-[5′-(3″-methoxy-4″-ethoxycarbonyloxyphenyl)-2′,4′-pentadienoyl]aminoethyl]4-diphenylmethoxy-piperidine (LINETASTINE),
3-[1-(4-chlorobenzyl)-3-(tert-butylthio)-5-isopropylindol-2-yl]-2,2-dimethylpropionic acid (MK-886),
(S)-N-[2-cyclohexyl-[(S)-(2-pyridyl)ethyl]-5-methylbenzoxazole-2-amine (ONTAZOLAST),
[4R-[4α(1E,3S*),5β]-1,4,5,6-tetrahydro-5-hydroxy-4-(3-hydroxy-1-octenyl)-1-phenylcyclopenta[b]pyrrole-2-pentanoic acid (PIRIPROST),
4-hydroxy-1-phenyl-3-(1-pyrrolidinyl)-1,8-naphthyriden-2(1H)-one (PIRODOMAST),
N-[3-(6-methyl-3-pyridyl)acryloxy]4-(4-diphenylmethyl-1-piperazinyl)butylamine (TAGORIZINE),
4,4-bis[4-(quinolin-2-ylmethoxy)phenyl]pentanoic acid (VML-530),
6-[3-fluoro-5-(4-methoxytetrahydropyran-4-yl)phenoxymethyl]-1-methylquinolin-2(1H)-one (ZD-2138) and
(+/−)-1-(1-benzo[b]thien-2-ylethyl)-1-hydroxyurea (ZILEUTON);
lipoxygenase inhibitors, such as, for example,
N-[3-[5-(4-fluorophenoxy)-2-furyl]-1-methyl-2-propynyl]-N-hydroxyurea (A-78773),
1-(6-phenoxy-2H-1-benzopyran-3-ylmethyl)-1-hydroxyurea (CGS-23885),
2,3,5-trimethyl-6-(12-hydroxy-5,10-dodecadiynyl)-1,4-benzoquinone (DOCEBENONE),
4-[[(6-hydroxy-4,5,7-trimethyl-2-benzothiazolyl)amino]methyl]benzenesulphonamide (E-6080),
N-[2-[4-(diphenylmethoxy)piperidin-1-yl]ethyl]-3-hydroxy-5-(3-pyridylmethoxy)naphthalene-2-carboxamide (NC-2000),
2-[3-(1-hydroxyhexyl)phenoxymethyl)quinoline (REV-5901A),
[2-[3,5-bis(tert-butyl)-4-hydroxyphenylthio]-1-methylpropoxy]acetic acid (SC-45662),
4-hydroxy-7-(4-hydroxy-3,5-dimethoxycinnamoylamino)-1-methyl-3-octyloxy-2(1H)-quinoline (TA-270),
3-[5-(4-chlorophenyl)-1-(4-methoxyphenyl)pyrazol-3-yl]-N-hydroxy-N-methylpropionamide (TEPOXALIN),
S-(+)-α-methyl-6-(2-quinolinylmethoxy)-2-naphthaleneacetic acid (WY-50295) and
(2S,4R)-5-[4-(4-hydroxy-2-methyltetrahydropyran-4-yl)-thien-2-yl-sulphanyl]-1-methyl-2,3-dihydro-1H-indol-2-one (ZD-4407);
inhibitors of mediator release, such as, for example,
N,N′-(2-chloro-5-cyano-m-phenylene)bis[glycolamide]diacetat (ACREOZAST),
1-[4-[3-[4-[bis(4-fluorophenyl)hydroxymethyl]-1-piperidinyl]propoxy]-3-methoxyphenyl]ethanone (AHR-5333B),
8-hexyloxy-3-(1H-tetrazol-5-yl)-2H-chromen-2-one (AL-136),
2-amino-7-isopropyl-5-oxo-5H-[1]benzopyrano[2,3-b]pyridine-3-carboxylic acid (AMLEXANOX),
4-(1H-tetrazol-5-yl)-N-[4-(1H-tetrazol-5-yl)pheny]benzamide (ANDOLAST),
2-ethoxyethyl N-[4-(3-methylisoxazol-5-yl)thiazol-2-yl]oxamate (ASOBAMAST),
3-[3-(methylcarbamoyloxy)propyl]-1-propylquinoxalin-2(1H)-one (BAMAQUIMAST),
4′-tert-butylphenyl trans-4-guanidinomethylcyclohexanecarboxylate (BATEBULAST),
6-butyryl-1-ethyl-4-hydroxy-7-methyl-2-oxo-1,2-dihydroquinoline-3-carboxylic acid (CGP-25875),
5-methoxy-3-isopropoxy-1-phenyl-N-(1H-tetrazol 5-yl)-1H-indole-2-carboxamide (CI-949),
3-isopropoxy-5-methoxy-N-(1H-tetrazol-5-yl)benzo[b]thiophene-2-carboxamide (CI-959),
diethyl 1,3-bis[2-(ethoxycarbonyl)₄-oxo-4H-benzo[b]pyran-5-yloxy]-2-propyl-L-lysinate (CROMOGLICATE LISETIL),
5,5′-(2-hydroxytrimethylenedioxy)bis(4-oxo-4H-1-benzopyran-2-carboxylic acid) (CROMOGLYCINIC ACID),
11-oxo-11H-pyrido[2,1-b]quinazoline-2-carboxylic acid (DOQUALAST),
1-[2-[(2,6-dimethyl-3-nitro-4-pyridyl)amino]ethyl]4-(diphenylmethyl)piperazine (ELBANIZINE),
6-(1-pyrrolidinyl)-N-(1H-tetrazol-5-yl)pyrazine-2-carboxamide (HSR-6071),
2-(ethoxymethyl)pteridin-4(3H)-one (LCB-2183),
1,6-dihydro-2-[2-(2-methylpropoxy)anilino]-6-oxo-5-pyrimidine-carboxylic acid (MAR-99),
4,6-dioxo-1-ethyl-10-propyl-4H,6H-pyrano[3,2-g]quinoline-2,8-dicarboxylic acid (NEDOCROMIL),
3-(5-methylfurfuryl)-2-(4-piperidylamino)-3H-imidazo[4,5-b]pyridine (NOBERASTINE),
1-[3-[4-(diphenylmethyl)-1-piperazinyl]propyl]-2-benzimidazolinone (OXATOMIDE),
9-methyl-3-(1H-tetrazol-5-yl)-4H-pyrido[1,2-a]pyrimidin-4-one (PEMIROLAST),
7-[3-[4-[(4-chlorophenyl)methyl]-1-piperazinyl]propoxy]-3,4-dimethyl-2H-1-benzopyran-2-one (PICUMAST),
9-[2-oxo-2-(pyrrolidin-1-yl)ethyl]-2,4-bis(pyrrolidin-1-yl)-9H-pyrimido[4,5-b]indole (PNU-142731A)
2-carbomethoxy-5chloro-1,3-oxazolo[4,5-h]quinoline (QUAZOLAST),
4-oxo-1-phenoxy-N-1H-tetrazol-5-yl-4H-quinolizine-3-carboxamide (QUINOTOLAST),
isoamyl 5,6-dihydro-7,8-dimethyl-4,5-dioxo-4H-pyrano[3,2-c]quinoline-2-carboxylate (REPIRINAST),
[2-[4-(3-ethoxy-2-hydroxopropoxy)phenylcarbamoyl]ethyl]dimethylsulphonium p-toluenesulphonate (SUPLATAST TOSILATE),
6-methyl-N-(1H-tetrazol-5-yl)-2-pyridine (TA-5707),
butyl N-[3-(1H-tetrazol-5-yl)phenyl]oxamate (TAZANOLAST),
ethyl 4-methoxyphenyl-4-thiazolyl-2-oxamate (TIOXAMAST) and
N-acetylaspartyl-glutamic acid magnesium salt (ZY-15106);
tachykinin NK, antagonists, such as, for example,
CP-122721,
(3aS,4S,7aS)-4-(2-methoxyphenyl)-2-[2(S)-(2-methoxyphenyl)-propionyl]-7,7-diphenylperhydroisoindol-4-ol (DAPITANT),
N-[3-(2-pentylphenyl)propanoyl]-threonyl-N-methyl-2,3-dehydrotyrosyl-leucyl-D-phenylalanyl-allothreonyl-asparaginyl-serine C-1.7-O-3.1-lactone (FK-224) and
1-[2-[3-(3,4-dichlorophenyl)-1-[2-(3-isopropoxyphenyl)acetyl]piperidin-3(S)-yl]ethyl]-4-phenyl-1-azoniabicyclo[2.2.2]octane chloride (NALPITANTIUM CHLORIDE);
tachykinin NK₂ antagonists, such as, for example,
(S)-N-[4-(4-acetamido-4-phenylpiperidin-1-yl)-2-(3,4-di-chlorophenyl)-butyi]-N-methylbenzamide (SAREDUTANT);
thromboxane A₂ antagonists, such as, for example,
4-[2-(4-chlorobenzenesulphonylamino)ethyl]benzeneacetic acid (DALTROBAN),
3-(1H-imidazol-1-ylmethyl)-2-methyl-1H-indole-1-propionic acid (DAZMEGREL),
(+)-(Z)-7-[3-endo-(phenylsulphonylamino)bicyclo[2.2.1]hept-2-exo-yl]heptenoic acid (DOMITROBAN),
7-[2α,4α-(dimethylmethano)-6-β-(2-cyclopentyl-2β-hydroxyacetamido)-1α-cyclohexyl]-5(Z)-heptenoic acid (ONO-3708) and
3-(4-tert-butylthiazol-2-ylmethoxy)-N-[5-[3-(4-chlorophenylsulphonyl)propyl]-2-(1H-tetrazol-5-ylmethoxy)phenyl]benzamide (YM-158);
thromboxane synthase inhibitors, such as, for example,
2-(1-imidazolylmethyl)-4,5-dihydrobenzo[b]thiophene-6-carboxylic acid (MITRODAST),
1-[3-[4-(diphenylmethyl)piperazin-1-yl]propyl]-3-(imidazol-1-ylmethyl)indole-6-carboxylic acid (KY-234),
(E)-3-[4-(1H-imidazol-1-ylmethyl)phenyl]-2-propenoic acid (OZAGREL) and
4-[α-hydroxy-5-(1-imidazolyl-2-methylbenzyl]-3,5-dimethylbenzoic acid (Y-20811);
α₄,β₁-(VLA-4) antagonists, such as, for example,
3-(1,3-benzodioxol-5-yl)-3-[N-[2-(4-hydroxyphenyl)acetyl]-D,L-leucyl-amino]propionic acid (BIO-1006),
N-[[4-[[[(2-methylphenyl)amino]carbonyl]amino]phenyl]acetyl]-L-leucyl-L-a-aspartyl-L-valyl-L-proline (BIO-1211),
N-[5,5-dimethyl-3-(4-methylphenylsulphonyl)thiazolidin-4(R)-ylcarbonyl]-4-O-[3-(dimethylamino)propyl]-L-tyrosine (CT-747),
N-(4-methylphenylsulphonyl)-L-prolyl-L-phenylalanine (CT-757),
N-(4-methylphenylsulphonyl)-L-prolyl-4-(4-piperidinylcarboxamido)-L-phenylalanine (CT-767),
N-[3-acetyl-4(S)-thiazolidinylcarbonyl]-L-[4-O-(2,6-dichlorobenzyl)]tyrosine (CT-5219),
1-methyl-4-[N-methyl-N-(2-phenylacetyl)-L-leucyl-L-aspartyl-L-phenylalanyl]piperazine (CY-9701),
3-1N-(3,4-dimethoxybenzyl)-N-[2-[2-[3-methoxy-4-[3-(2-methylphenyl)ureido]phenyl]acetamido]-acetyl]amino]propionic acid (IVL-745),
3(R)-[1-[2-[4-[3-(2-methylphenyl)ureido]phenyl]acetyl]pyrrolidin-2(S)-ylcarboxamido]butyric acid (OMEPUPA-V),
3(S)-(1,3-benzodioxol-5-yl)-3-[3-[2-(benzylsulphanyl)-1(S)-(phenylsulphanylmethyl)ethyl]ureido]-propionic acid (TBC-3342),
3(S)-(1,3-benzodioxol-5-yl)-3-[N3-[1(S)-[N,N-bis(2-thienylmethyl)carbamoyl]pentyl]ureido]propionic acid (TBC-3486),
N-[3(R)-carboxy-2,2,3-trimethylcyclopent-1(S)-ylcarbonyl]4-(2,6-dichlorobenzamido)-L-phenylalanine (TR-9109),
2(S)-(2,6-dichlorobenzamido)-3-(2′,6′-dimethoxybiphenyl-4-yl)-propionic acid (TR-14035) and
3-[4-(4-carbamoylpiperidin-1-ylcarbonyloxy)phenyl]-2(S)-[4-methyl-2(S)-[2-(2-methylphenoxy)acetamido]pentanamido]-propionic acid;
VCAM inhibitors, such as, for example,
4-(4-bromophenoxy)thieno[2,3-c]pyridine-2-carboxamide (A-249377),
4-(4-bromophenoxy)-N-methylthieno[2,3-c]pyridine-2-carboxamide (A-277232),
N-methyl-4-[4-(trifluoromethyl)phenoxy]thieno[2,3-c]pyridine-2-carboxamide (A-277249),
1-[2-[2,3-dichloro-4-[trans-2-[N-[3-(2-oxopyrrolidin-1-yl)propyl]carbamoyl]cyclopropyl]phenylsulphanyl]-phenyl]piperidine-3-carboxylic acid (A-324920),
5(R)-(4-bromobenzyl)-3-(3,5-dichlorophenyl)-1,5-dimethylimidazolidine-2,4-dione (BIRT-377) and
N-(phenylsulphonyl)-4(S)-phenyl-L-prolyl-4-(2,6-dimethoxyphenyl)-L-phenylalanine (TR-14531), and
chimase inhibitors, such as, for example,
3-carboxyphenylmethyl(6R,7R)-7-methoxy-7-[(2-methoxybenzoyl)amino]-3-[[(1-methyl-1H-tetrazol-5-yl)thio]methyl]-8-oxo-5-oxa-1-azabicyclo[4.2.0]oct-2-en-2-carboxylate (B-135),
4-carboxyphenylmethyl(6R,7R)-7-methoxy-7-[(2-methoxybenzoyl)amino]-3-[[(1-methyl-1H-tetrazol-5-yl)thio]methyl]-8-oxo-5-oxa-1-azabicyclo[4.2.0]oct-2-en-2-carboxylate (B-136),
3-methylphenylmethyl(6R,7R)-7-methoxy-7-[(2-methoxybenzoyl)amino]-8-oxo-3-[[[1-[2-oxo-2-(2-propenyloxy)ethyl]-1H-tetrazol-5-yl]thio]methyl]-5-oxa-1-azabicyclo[4.2.0]oct-2-ene-2-carboxylate (B-146),
3-methylbenzyl(6R,7R)-3-[1-(carboxymethyl)tetrazol-5-ylsulphanylmethyl]-7-methoxy-7-(2-methoxybenzamido)-1-oxa-3-cephem-4-carboxylate (B-152) and 3-methylbenzyl(6R,7R)-3-[1-(carboxymethyl)tetrazol-5-ylsulphanylmethyl]-7-methoxy-7-(2-ethoxybenzamido)-1-oxa-3-cephem-4-carboxylate (B-153).

The airway therapeutics can be present as such or in chemically bonded form. It is understood hereby that the active compounds mentioned can also be present, for example, in the form of their pharmacologically acceptable salts and/or as solvates (e.g. hydrates), and/or in the form of their N-oxides etc. Suitable pharmacologically acceptable salts here are in particular water-soluble and water-insoluble acid addition salts with acids such as, for example, hydrochloric acid, hydrobromic acid, phosphoric acid, nitric acid, sulphuric acid, acetic acid, citric acid, D-gluconic acid, benzoic acid, 2-(4-hydroxybenzoyl)benzoic acid, butyric acid, sulphosalicylic acid, maleic acid, lauric acid, malic acid, fumaric acid, succinic acid, oxalic acid, tartaric acid, embonic acid, stearic acid, toluenesulphonic acid, methanesulphonic acid or 1-hydroxy-2-naphthoic acid, the acids being employed in salt preparation—depending on whether it is a mono- or polybasic acid and depending on which salt is desired—in an equimolar quantitative ratio or one differing therefrom. Furthermore, the active compounds mentioned can also be present as pure enantiomers or as enantiomer mixtures in any mixing ratio.

Airway therapeutics to be emphasized as being suitable for combined application with a reversible proton pump inhibitor in the meaning of the invention are in particular
from the class of the β₂-adrenoceptor agonists the active compounds
BAMBUTEROL, BITOLTEROL, BROXATEROL, CARBUTEROL, DOPEXAMINE, DROPRENILAMINE, FORMOTEROL, LEVOSALBUTAMOL, MABUTEROL, PIRBUTEROL, REPROTEROL, SALBUTAMOL, SALMETEROL, TERBUTALINE, TIARAMIDE and TULOBUTEROL;
from the class of the muscarinic receptor antagonists the active compounds
FLUTROPIUM BROMIDE, IPRATROPIUM BROMIDE, OXITROPIUM BROMIDE and TIOTROPIUM BROMIDE;
from the class of the theophylline-like bronchodilators the active compounds
AMINOPHYLLINE, DIPROPHYLLINE, DOXOFYLLINE, OXYFEDRINE, PENTIFYLLINE, PENTOXIFYLLINE, PROPENTOFYLLINE and PROXYPHYLLINE;
from the class of the PDE3/4- and PDE4 inhibitors the active compounds
(Z)-3-(3,5-dichloro-4-pyridyl)-2-[4-(2-indanyloxy-5-methoxy-2-pyridyl]propenenitrile,
N-[9-amino-4-oxo-1-phenyl-3,4,6,7-tetrahydropyrrolo[3,2,1-jk][1,4]benzodiazepin-3(R)-yl]pyridine-3-carboxamide (CI-1044),
N-(3,5-dichloro-4-pyridinyl-2-[1-(4-fluorobenzyl)-5-hydroxy-1H-indol-3-yl]-2-oxoacetamide (AWD-12-281),
cis-[4-cyano-4-(3-cyclopentyloxy-4-methoxyphenyl)cyclohexan-1-carboxylic acid (CILOMILAST),
8-amino-1,3-bis(cyclopropylmethyl)xanthine (CIPAMFYLLINE),
2-methyl-1-[2-(1-methylethyl)pyrazolo[1,5-a]pyridin-3-yl]-1-propanone (IBUDILAST),
2-(2,4-dichlorophenylcarbonyl)-3-ureidobenzofuran-6-yl methanesulphonate (LIRIMILAST),
(−)-cis-9-ethoxy-8-methoxy-2-methyl-1,2,3,4,4a,10b-hexahydro-6-(4-diisopropylaminocarbonylphenyl)-benzo[c][1,6]naphthyridine (PUMAFENTRINE),
3-(cyclopropylmethoxy)-N-(3,5-dichloro-4-pyridyl)-4-(difluoromethoxy)benzamide (ROFLUMILAST), ROFLUMILAST-N-OXIDE and
3-[[3-(cyclopentyloxy)-4-methoxyphenyl]methyl]-N-ethyl-8-(1-methylethyl)-3H-purine-6-amine (V-11294A);
from the class of the cysteinyl-leukotriene, receptor antagonists the active compounds
2-[1-[1(R)-[3-[2(E)-(7-chloroquinolin-2-yl)vinyl]phenyl]-3-[2-(1-hydroxy-1-methylethyl)phenyl]propylsulphanylmethyl]-cyclopropyl]acetic acid (MONTELUKAST),
8-[4-(4-phenylbutoxy)benzamido]-2-(tetrazol-5-yl)-4H-1-benzopyran-4-one (PRANLUKAST) and
4-(5-cyclopentyloxycarbonylamino-1-methylindol-3-yl-methyl)-3-methoxy-N-o-tolylsulphonylbenzamide (ZAFIRLUKAST),
from the class of the leukotriene synthesis inhibitors the active compound
(+/−)-1-(1-benzo[b]thien-2-ylethyl)-1-hydroxyurea (ZILEUTON);
from the class of the lipoxygenase inhibitors the active compound
3-[5-(4-chlorophenyl)-1-(4-methoxyphenyl)pyrazol-3-yl]-N-hydroxy-N-methylpropionamide (TEPOXALIN),
from the class of the inhibitors of mediator release the active compounds
2-amino-7-isopropyl-5-oxo-5H-[1]benzopyrano[2,3-b]pyridine-3-carboxylic acid (AMLEXANOX),
5,5′-(2-hydroxytrimethylenedioxy)bis(4-oxo-4H-1-benzopyran-2-carboxylic acid) (CROMOGLYCINIC ACID),
4,6-dioxo-1-ethyl-10-propyl-4H,6H-pyrano[3,2-g]quinoline-2,8-dicarboxylic acid (NEDOCROMIL),
1-[3-[4-(diphenylmethyl)-1-piperazinyl]propyl]-2-benzimidazolinone (OXATOMIDE),
9-methyl-3-(1H-tetrazol-5-yl)-4H-pyrido[1,2-a]pyrimidin-4-one (PEMIROLAST),
isoamyl 5,6-dihydro-7,8-dimethyl-4,5-dioxo-4H-pyrano[3,2-c]quinoline-2-carboxylate (REPIRINAST),
[2-[4-(3-ethoxy-2-hydroxopropoxy)phenylcarbamoyl]ethyl]dimethylsulphonium p-toluenesulphonate (SUPLATAST TOSILATE) and
butyl N-[3-(1H-tetrazol-5-yl)phenyl]oxamate (TAZANOLAST),
from the class of the thromboxane A₂ antagonists the active compound
(+)-(Z)-7-[3-endo-(phenylsulphonylamino)bicyclo[2.2.1]hept-2-exo-yl]heptenoic acid (DOMITROBAN),
and from the class of the thromboxane synthase inhibitors the active compound
(E)-3-[4-(1H-imidazol-1-ylmethyl)phenyl]-2-propenoic acid (OZAGREL).

The invention provides especially the combined use of reversible proton pump inhibitors and airway therapeutics from the class of the PDE3/4- and PDE4 inhibitors for the treatment of airway disorders.

The invention furthermore provides especially the combined use of reversible proton pump inhibitors and CICLESONIDE for the treatment of airway disorders.

The invention provides particularly especially the combined use of a reversible proton pump inhibitor selected from the group consisting of AG-2000, AU-461, BY112, Soraprazan, CP-113411, DBM-819, KR-60436, pumaprazole, SKF-96067, SKF-96356, SKF-97574, T-330, T-776, WY-27198, YH-1885, YJA-20379-8 and YM-19020 and an airway therapeutic from the class of the PDE3/4- and PDE4 inhibitors selected from the group consisting of (Z)-3-(3,5-dichloro-4-pyridyl)-2-[4-(2-indanyloxy-5-methoxy-2-pyridyl]propenenitrile, N-[9-amino-4-oxo-1-phenyl-3,4,6,7-tetrahydropyrrolo[3,2,1-jk][1,4]benzodiazepine-3(R)-yl]pyridine-3-carboxamide (CI-1044), N-(3,5-dichloro-4-pyridinyl)-2-[1-(4-fluorobenzyl)-5-hydroxy-1H-indol-3-yl]-2-oxoacetamide (AWD-12-281), cis-[4-cyano-4-(3-cyclopentyloxy-4-methoxyphenyl)cyclohexane-1-carboxylic acid (CILOMILAST), 8-amino-1,3-bis(cyclopropylmethyl)xanthine (CIPAMFYLLINE), 2-methyl-1-[2-(1-methylethyl)pyrazolo[1,5-a]pyridin-3-yl]-1-propanone (IBUDILAST), 2-(2,4-dichlorophenylcarbonyl-3-ureidobenzofuran-6-yl methanesulphonate (LIRIMILAST), (−)-cis-9-ethoxy-8-methoxy-2-methyl-1,2,3,4,4a,10b-hexahydro-6-(4-diisopropylaminocarbonylphenyl)benzo[c]-[1,6]naphthyridine (PUMAFENTRIN E), 3-(cyclopropylmethoxy)-N-(3,5-dichloro-4-pyridyl)-4-(difluoromethoxy)benzamide (ROFLUMILAST), ROFLUMILAST-N-OXIDE and 3-[[3-(cyclopentyloxy)-4-methoxyphenyl]methyl]-N-ethyl-8-(1-methylethyl)-3H-purine-6-amine (V-11294A) for the treatment of airway disorders.

The invention preferably provides the combined use of a reversible proton pump inhibitor selected from the group consisting of AU-461, Soraprazan, DBM-819, KR-60436, T-330, YH-1885 and YJA-20379-8 and an airway therapeutic from the class of the PDE3/4- and PDE4 inhibitors selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo[1,5-a]pyridin-3-yl]-1-propanone (IBUDILAST), (−)cis-9-ethoxy-8-methoxy-2-methyl-1,2,3,4,4a,10b-hexahydro-6-(4-diisopropylaminocarbonyl-phenyl)-benzo[c][1,6]naphthyridine (PUMAFENTRINE), 3-(cyclopropylmethoxy)-N-(3,5-dichloro-4-pyridyl)-4-(difluoromethoxy)benzamide (ROFLUMILAST) and ROFLUMILAST-N-OXIDE for the treatment of airway disorders.

The invention furthermore preferably provides the combined use of a reversible proton pump inhibitor selected from the group consisting of AU-461, Soraprazan, DBM-819, KR-60436, T-330, YH-1885 and YJA-20379-8 and CICLESONIDE for the treatment of airway disorders.

The invention particularly preferably provides the combined use of an APA selected from the group consisting of (7R,8R,9R-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethylimidazo[1,2-a]pyridine-6-carboxylic acid (2-hydroxy-ethyl)-amide and 8-(2-ethyl-6-methyl-benzylamino)-2,3-dimethyl-imidazo[1,2-a]pyridine-6-carboxylic acid amide, or a salt of such APA, and 3-(cyclopropylmethoxy)-N-(3,5-dichloro-4-pyridyl)-4-difluoromethoxy)benzamide (ROFLUMILAST) or ROFLUMILAST-N-OXIDE for the treatment of airway disorders.

The invention furthermore particularly preferably provides the combined use of an APA selected from the group consisting of (7R,8R,9R-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy}2,3-dimethyl-1-(2-methyl-cyclopropylmethyl)-1H-pyrrolo[2,3-d]pyridazine, 8-(2,6-dimethyl-benzylamino)-2,3-dimethylimidazo[1,2-a]pyridine-6-carboxylic acid (2-hydroxy-ethyl)-amide and 8-(2-ethyl-6-methyl-benzylamino)-2,3-dimethyl-imidazo[1,2-a]pyridine-6-carboxylic acid amide, or a salt of such APA, and CICLESONIDE for the treatment of airway disorders.

In a particularly preferred embodiment, the invention provides the combined use of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydropyrano[2,3-c]imidazo[1,2-a]pyridine or (7R,8R,9R)-7-(2-methoxyethoxy)-2,3-dimethyl-8-(N,N-dimethylaminomethylcarbonyloxy)-9phenyl-7,8,9,10-tetrahydroimidazo[1,2-h][1,7]naphthyridine, or of a salt thereof, and 3-(cyclopropylmethoxy)-N-(3,5-dichloro-4-pyridyl)-4-(difluoromethoxy)benzamide (ROFLUMILAST) or ROFLUMILAST-N-OXIDE for the treatment of airway disorders.

In a further particularly preferred embodiment, the invention provides the combined use of (7R,8R,9R)-2,3-dimethyl-8-hydroxy-7-(2-methoxyethoxy)-9-phenyl-7,8,9,10-tetrahydroimidazo-[1,2-h]-[1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine or (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, or of a salt thereof, and CICLESONIDE for the treatment of airway disorders.

In a very particularly preferred embodiment, the invention provides the combined use of (7R,8R,9R)-2,3-dimethyl-8-hydroxy-7-(2-methoxyethoxy)-9-phenyl-7,8,9,10-tetrahydroimidazo-[1,2-h]-[1,7]naphthyridine (Soraprazan) or of a salt thereof, and 3-(cyclopropylmethoxy)-N-(3,5-dichloro-4-pyridyl)-4-(difluoromethoxy)benzamide (ROFLUMILAST) for the treatment of airway disorders.

In a further very particularly preferred embodiment, the invention provides the combined use of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan) or of a salt thereof, and CICLESONIDE for the treatment of airway disorders.

Airway disorders which may be mentioned are in particular allergen- and inflammation-induced pulmonary abnormalities and bronchial disorders (for example bronchitis, obstructive bronchitis including COPD, spastic bronchitis, allergic bronchitis, allergic asthma, bronchial asthma, in particular night-time asthma attacks, pneumonitis and pulmonary fibrosis), which can be treated by the combination according to the invention also in the context of a long-term therapy (if desired with appropriate adjustment of the dose of the individual components to the needs at the time, for example needs subject to seasonally related variations).

“Combined use” or “combination” within the meaning of the present invention is to be understood as meaning that the individual components can be administered simultaneously (in the form of a combination medicament), more or less simultaneously (from separate pack units) or in succession (one directly after the other or else alternatively within a relatively large time span) in a manner which is known per se and customary.

Within the meaning of the present invention, “use” is preferably understood as meaning the oral administration of both active compounds. However, it is also conceivable to administer the reversible proton pump inhibitor parenterally (for example intravenously) and/or to administer the airway therapeutic parenterally or topically (in particular by inhalation). For administration by inhalation, the airway therapeutic is preferably administered in the form of an aerosol, the aerosol particles of solid, liquid or mixed composition having a diameter of 0.5 to 10 μm, advantageously of 2 to 6 μm.

Aerosol generation can be carried out, for example, by pressure-operated jet atomizers or ultrasonic atomizers, but advantageously by propellant-operated metered aerosols or propellant-free administration of micronized active compounds from inhalation capsules.

Depending on the inhaler system used, in addition to the active compounds the administration forms also contain the required excipients, such as, for example, propellants (e.g. Frigen in the case of metered aerosols), surface-active substances, emulsifiers, stabilizers, preservatives, flavourings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as appropriate as possible for the patient. In addition to the use of adaptors (spacers, expanders) and pear-shaped containers (e.g. Nebulator®, Volumatic®), and automatic devices emitting a puff of spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

The active compounds are dosed in an order of magnitude customary for the individual dosage, where it may be possible, on account of the individual actions, which are mutually positively influencing and reinforcing, to reduce the respective dosages on the combined administration of the active compounds compared with the norm, or where—if the dosage of the individual compounds is the customary dosage—a surprising better and longer-lasting activity is obtained.

The reversible proton pump inhibitor is usually administered in a daily dose of in particular 0.1 to 1.5 mg/kg of body weight, if appropriate in the form of a plurality, preferably 1 to 2, individual doses, to obtain the desired result. In the case of the airway therapeutics, the dose customary for the person skilled in the art is administered, which, depending on the class of active compound, may vary within a very broad range. Thus, for example, the β2 adrenoceptor agonist is—depending on the active compound—in the case of administration by inhalation usually administered in a dosage of, for example, 0.002 to 2.0 mg per day. For the POE inhibitors, it is possible in the case of oral administration to vary the doses—depending on the active compound—within a wide range, it being possible, as a framework, to start from a dose of 1-2000 μg/kg of body weight. In the case of the administration of the preferred POE inhibitor roflumilast, the dosage is in the range from 2-20 μg/kg of body weight.

The reversible proton pump inhibitors or airway therapeutics to be administered orally are formulated—if appropriate jointly—to give medicaments according to processes known per se and familiar to the person skilled in the art. The pharmacologically active compounds are employed as medicaments, preferably in combination with suitable pharmaceutical excipients or vehicles, in the form of tablets, coated tablets, capsules, emulsions, suspensions or solutions, the active compound content advantageously being between 0.1 and 95% and, by the appropriate choice of the excipients and vehicles, it being possible to achieve a pharmaceutical administration form precisely tailored to the active compound(s) and/or to the desired onset of action (e.g. a sustained-release form or an enteric form). The person skilled in the art is familiar on the basis of his/her expert knowledge with which excipients or vehicles are suitable for the desired pharmaceutical formulations. In addition to solvents, gel-forming agents, tablet excipients and other active compound carriers, it is possible to use, for example, antioxidants, dispersants, emulsifiers, antifoams, flavour corrigents, preservatives, solubilizers, colorants or permeation promoters and complexing agents (e.g. cyclodextrins).

In a further aspect, the invention provides the use of a reversible proton pump inhibitor in combination with an airway therapeutic for treating patients suffering from an airway disorder.

The invention further provides a method for treating airway disorders which comprises administering to a patient in need of such a treatment an effective amount of a reversible proton pump inhibitor together with an airway therapeutic.

The invention further provides the use of reversible proton pump inhibitors and airway therapeutics for preparing combination medicaments for treating airway disorders.

The invention further provides a pharmaceutical preparation for treating airway disorders, which preparation comprises, as active compounds, a reversible proton pump inhibitor and an airway therapeutic.

The invention further provides a ready-to-use medicament, comprising, as active compounds, a reversible proton pump inhibitor and an airway therapeutic, which contains a reference to the fact that these active compounds are to be taken for the treatment of an airway disorder more or less simultaneously or in succession (one directly after the other or else within a relatively large time span).

The invention further provides a ready-to-use medicament, comprising, as active compound, a reversible proton pump inhibitor, which contains a reference to the fact that this reversible proton pump inhibitor is to be taken for the treatment of an airway disorder more or less simultaneously or in succession (one directly after the other or else within a relatively large time span) with an airway therapeutic.

The invention further provides to a ready-to-use medicament, comprising, as active compound, an airway therapeutic, which contains a reference to the fact that this airway therapeutic is to be taken for the treatment of an airway disorder more or less simultaneously or in succession (one directly after the other or else within a relatively large time span) with a reversible proton pump inhibitor.

1. A pharmaceutical composition comprising a reversible proton pump inhibitor and an airway therapeutic in fixed or free combination.

2. A pharmaceutical composition according to claim 1 in fixed oral combination.

3. A method for treating an airway disorder in a patient comprising administering to a patient in need of such a treatment an effective amount of a reversible proton pump inhibitor together with an airway therapeutic.

4. A method according to claim 3, wherein the airway disorder is selected from the group consisting of bronchitis, obstructive bronchitis, COPD, spastic bronchitis, allergic bronchitis, allergic asthma, bronchial asthma, pneumonitis and pulmonary fibrosis.

5. A pharmaceutical preparation comprising as active compounds, a reversible proton pump inhibitor and an airway therapeutic.

6. A ready-to-use medicament, comprising, as active compounds, a reversible proton pump inhibitor and an airway therapeutic, which contains a reference to the fact that said active compounds are to be taken for the treatment of an airway disorder more or less simultaneously or in succession.

7. A ready-to-use medicament, comprising, as active compound, a reversible proton pump inhibitor, which contains a reference to the fact that said reversible proton pump inhibitor is to be taken for the treatment of an airway disorder more or less simultaneously or in succession with an airway therapeutic.

8. A ready-to-use medicament, comprising, as active compound, an airway therapeutic, which contains a reference to the fact that said airway therapeutic is to be taken for the treatment of an airway disorder more or less simultaneously or in succession with a reversible proton pump inhibitor.

9. A pharmaceutical composition according to claim 1, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxy-ethoxy)-9-phenyl-7,8,9,10-tetrahydroimidazo[1,2-h][1,7]naphthyridine and the hydrates, solvates, salts, hydrates of the salts and solvates of the salts thereof.

10. A pharmaceutical composition according to claim 1, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylaminocarbonylphenyl)benzo[c][1,6]naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

11. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

12. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

13. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h](1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

14. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

15. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

16. A pharmaceutical composition according to claim 1, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.

17. A method according to claim 3, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxy-ethoxy)-9-phenyl-7,8,9,10-tetrahydroimidazo[1,2-h][1,7]naphthyridine and the hydrates, solvates, salts, hydrates of the salts and solvates of the salts thereof.

18. A method according to claim 3, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylamino-carbonylphenyl)benzo[c][1,6]-naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

19. A method according to claim 3, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

20. A method according to claim 3, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

21. A method according to claim 3, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

22. A method according to claim 3, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

23. A method according to claim 3, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

24. A method according to claim 3, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.

25. A pharmaceutical preparation according to claim 5, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxyethoxy)-9-phenyl-7,8,9,10-tetrahydroimidazo[1,2-h][1,7]naphthyridine and the hydrates, solvates, salts, hydrates of the salts and solvates of the salts thereof.

26. A pharmaceutical preparation according to claim 5, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylamino-carbonylphenyl)benzo[c][1,6]-naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

27. A pharmaceutical preparation according to claim 5, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

28. A pharmaceutical preparation according to claim 5, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

29. A pharmaceutical preparation according to claim 5, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

30. A pharmaceutical preparation according to claim 5, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

31. A pharmaceutical preparation according to claim 5, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

32. A pharmaceutical preparation according to claim 5, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.

33. A ready-to-use medicament according to claim 6, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxyethoxy)-9-phenyl-7,8,9,10-tetrahydroimidazo[1,2-h][1,7]naphthyridine and the hydrates, solvates, salts, hydrates of the salts and solvates of the salts thereof.

34. A ready-to-use medicament according to claim 6, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylamino-carbonylphenyl)benzo[c][1,6]-naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

35. A ready-to-use medicament according to claim 6, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

36. A ready-to-use medicament according to claim 6, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

37. A ready-to-use medicament according to claim 6, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

38. A ready-to-use medicament according to claim 6, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

39. A ready-to-use medicament according to claim 6, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

40. A ready-to-use medicament according to claim 6, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.

41. A ready-to-use medicament according to claim 7, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxyethoxy)-9-phenyl-7,8,9,10-tetrahydroimidazo[1,2-h][1,7]naphthyridine and the hydrates, solvates, salts, hydrates of the salts and solvates of the salts thereof.

42. A ready-to-use medicament according to claim 7, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylamino-carbonylphenyl)benzo[c][1,6]-naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

43. A ready-to-use medicament according to claim 7, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

44. A ready-to-use medicament according to claim 7, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

45. A ready-to-use medicament according to claim 7, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c)imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

46. A ready-to-use medicament according to claim 7, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

47. A ready-to-use medicament according to claim 7, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

48. A ready-to-use medicament according to claim 7, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.

49. A ready-to-use medicament according to claim 8, wherein the reversible proton pump inhibitor is selected from the group consisting of AU-461, BY359, DBM-819, KR-60436, T-330, YH-1885, YJA-20379-8 and (7R,8R,9R)-2,3-dimethyl-8-hydroxy-7-(2-methoxyethoxy)-9-phenyl-7,8,9,10-tetrahydroimidazo[1,2-h][1,7]naphthyridine and the hydrates, solvates, salts, hydrates of the salts and solvates of the salts thereof.

50. A ready-to-use medicament according to claim 8, wherein the airway therapeutic is selected from the group consisting of cis-[4-cyano-4-(3-cyclopentyloxy-4-methoxyphenyl)cyclohexane-1-carboxylic acid (CILOMILAST), 2-methyl-1-[2-(1-methylethyl)pyrazolo-[1,5-a]pyridin-3-yl]-1-propanone (IBUDILAST), (−)-cis-9-ethoxy-8-methoxy-2-methyl-1,2,3,4,4a,10b-hexahydro-6-(4-diisopropylamino-carbonylphenyl)benzo[c][1,6]-naphthyridine (PUMAFENTRINE), 3-(cyclopropylmethoxy)-N-(3,5-dichloro-4-pyridyl)-4-difluoromethoxy)benzamide (ROFLUMILAST), ROFLUMILAST N-OXIDE and the hydrates, solvates, salts, hydrates of the salts and solvates of the salts thereof.

51. A ready-to-use medicament according to claim 8, wherein the reversible proton pump inhibitor is selected from the group consisting of (7R,8R,9R)-2,3-dimethyl-8-hydroxy-7-(2-methoxyethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine, 7-(4-fluoro-benzyloxy)-2,3-dimethyl-1-(2-methyl-cyclopropylmethyl)-1H-pyrrolo[2,3-d]pyridazine, 7-(4-chloro-benzyloxy)-2,3-dimethyl-1-(2-methyl-cyclopropylmethyl)-1H-pyrrolo[2,3-d]pyridazine, 7-(2,4-difluoro-benzyloxy)-2,3-dimethyl-1-(2-methyl-cyclopropylmethyl)-1H-pyrrolo[2,3-d]pyridazine, 8-(2,6-dimethyl-benzylamino)-2,3-dimethyl-imidazo[1,2-a]pyridine-6-carboxylic acid (2-hydroxy-ethyl)-amide, 8-(2-ethyl-6-methyl-benzylamino)-2,3-dimethylimidazo[1,2-a]pyridine-6-carboxylic acid amide and the hydrates, solvates, salts, hydrates of the salts and solvates of the salts thereof.

52. A ready-to-use medicament according to claim 8, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof.

53. A ready-to-use medicament according to claim 8, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is CICLESONIDE or a hydrate, solvate, salt, hydrate of a salt or solvate of a salt thereof.

54. A ready-to-use medicament according to claim 8, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan), (7R,8R,9R)-2,3-dimethyl-8-hydroxy-7-(2-methoxyethoxy)-9-phenyl-7H-8,9-dihydro-pyrano[2,3-c]imidazo[1,2-a]pyridine, (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine and the hydrates, solvates, salts, hydrates of the salts and solvates of the salts thereof, and wherein the airway therapeutic is 3-(cyclopropylmethoxy)-N-(3,5-dichloro-4-pyridyl)-4-(difluoromethoxy)benzamide (ROFLUMILAST), ROFLUMILAST-N-OXIDE or a hydrate, solvate, salt, hydrate of a salt or solvate of a salt thereof.

55. A ready-to-use medicament according to claim 8, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is 3-(cyclopropylmethoxy)-N-(3,5-dichloro-4-pyridyl)-4-(difluoromethoxy)benzamide (ROFLUMILAST) or a hydrate, solvate, salt, hydrate of a salt or solvate of a salt thereof.

56. A ready-to-use medicament according to claim 8, wherein the reversible proton pump inhibitor is selected from the group consisting of (7R,8R,9R)-2,3-dimethyl-8-hydroxy-7-(2-methoxy-ethoxy)-9-phenyl-7,8,9,10-tetrahydroimidazo-[1,2-h][1,7]naphthyridine (Soraprazan) or a hydrate, solvate, salt, hydrate of a salt or solvate of a salt thereof, and wherein the airway therapeutic is CICLESONIDE or a hydrate, solvate, salt, hydrate of a salt or solvate of a salt thereof.
